(19) United States
(12) Plant Patent Application Publication
(10) Pub. No.: US 2002/0073462 P1
(43) Pub. Date: Jun. 13, 2002

(54) VARIETY OF GERANIUM PLANT NAMED 'FIWOPINK'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,008

(22) Filed: Dec. 8, 2000

(51) Int. Cl. A01H 5/00

(52) U.S. Cl. PLT/328

(57) ABSTRACT

A new and distinct zonal geranium plant with unusual shaped red-purple colored flowers above medium green foliage. The plant is suitable for pot planting or as a bedding plant.

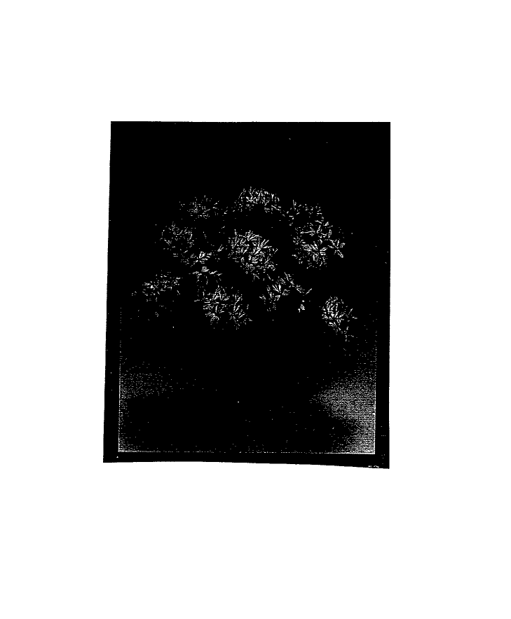

### BACKGROUND OF THE INVENTION

The present invention comprises a new and distinct cultivar of Pelargonium×hortorum known by the varietal name ‘Fiwopink’. The new variety was discovered in 1998 in a selective breeding program in Dresden, Germany. The new variety is the result of the crossing of three breeding varieties. The new variety was first asexually reproduced in 1999 by cuttings in Dresden, Germany. The new variety has been trial and field tested and has been found to retain its distinctive characteristics and remain true to type through successive propagations.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in diffuse light during summer. Color references are primarily to the R.H.S. Colour Chart of The Royal Horticultural Society of London.

Plant:

Classification:

Botanical.—Pelargonium×hortorum.

Commercial.—Zonal geranium.

Form.—Erect.

Height from media surface to top of foliage.—35-38 cm.

Height from media surface to top of umbel.—50 cm.

Growth.—Very high degree of branching.

Strength.—Does not require artificial support.

Response time.—3.5 weeks.

Leaves:

Size.—6-10 cm long.

Shape.—Stellar-type.

Margin.—Toothed.

Texture.—Smooth; some pubescence.

Color.—Green Group 139C with no zone.

Ribs and veins:

Color.—Darker than leaf surface.

Petioles:

Color.—Light green.

Length.—3-5 cm.

Stem/branches:

Color.—Medium green.

Internode length.—3-4 cm.

The bud:

Shape when just showing color:

Overall.—Semi-spherical.

Individual bud.—Narrow; elliptical.

Color.—Green Group 143C.

Size when just showing color:

Umbel.—7.5-9.5 cm across.

Individual bud.—1-1.5 cm long; 0.7 cm wide.

Inflorescence:

Blooming habit.—Star-like; single.

Size of fully open bloom.—2-3 cm.

Borne.—Flower on umbel; umbel on pedicel; pedicel on peduncle.

Flowers:

Form.—Zygomorph.

Number of petals.—5.

Size:

Upper petals.—0.4-0.6 cm wide; 1.5 cm long.

Lower petals.—0.6-1.0 cm wide; 1.5 cm long.

Petals:

Color.—

Upper surface.—Red-Purple Group 57D.

Lower surface.—Red-Purple Group 57D.

Eye.—Red-Purple Group 62C.

Texture and appearance.—Velvety.

Petaloids.—Absent.

Pedicel:

Length.—2.0-2.7 cm.

Color.—Green-reddish.

Peduncle:

Length.—12-14 cm.

Color.—Medium green.

Disease/pest resistance.—No unusual susceptibility to diseases or pests has been noted to date.

Lasting Quality.—Long-lasting; 3-4 weeks per umbel.

Reproductive organs:

Stamens:

Anthers.—3 mm long.

Filaments:

Length.—0.4 cm maximum.

Color.—Pink.

Pollen.—Orange.

Pistils:

Number.—Five.

Length.—0.3-0.4 cm.

Stigma.—Dark pink.

Style.—0.8 cm long.

Ovaries:

Color.—Greenish.

Length.—0.4 cm.

Fruit.—Present with 5 seeds; fruit production very weak.

### GENERAL CHARACTERISTICS

1. Stellar-shaped foliage.

2. Erect, compact growth.

3. Good branching.

4. Flowers are single with unique shape.

5. Long-lasting and highly resistant against rain.

6. Low care requirements.

I claim:

1. A new and distinct variety of geranium plant substantially as shown and described.
